(19) United States
(12) Patent Application Publication
(10) Pub. No.: US 2011/0301426 A1
(43) Pub. Date: Dec. 8, 2011

(54) Method and device for conditioning display of physiological parameter estimates on conformance with expectations

(76) Inventors: Yongji Fu, Vancouver, WA (US); Bryan Severt Hallberg, Vancouver, WA (US)

(21) Appl. No.: 12/802,331

(22) Filed: Jun. 4, 2010

(51) Int. Cl. A61B 5/00

(52) U.S. Cl. 600/300

(57) ABSTRACT

Method and device for continual physiological monitoring in which the display of physiological parameter estimates is conditioned on conformance of the estimates with expectations. Current estimates of physiological parameters are compared with expectations for the current estimates determined using prior estimates of the physiological parameters. Nonconformance with expectations can result in display of information indicating present unavailability of an estimate for the physiological parameter. The method and device are adaptable for use with various types of monitored physiological parameters and various expectation metrics.

### BACKGROUND OF THE INVENTION

This invention relates to physiological monitoring and, more particularly, to reducing unreliable physiological parameter output in physiological monitoring applications.

Continual monitoring of the physiological state of people who suffer from chronic diseases is an important aspect of chronic disease management. By way of example, continual respiratory monitoring is in widespread use in managing respiratory diseases, such as asthma. Continual monitoring of physiological state is also widely used in other contexts, such as elder care.

One serious problem encountered in continual physiological monitoring is parameter estimation error. Continual monitoring is often performed using a portable (e.g. wearable) device that continually acquires and analyzes a physiological signal, such as a signal that includes heart and lung sounds, as a person wearing the device goes about his or her daily life. The physiological signal acquired by the device can be rendered temporarily unreliable due to, for example, noise effects, motion effects, poor network connection and sensor malfunction. This can result in erroneous estimation of physiological parameters by the device and outputting of erroneous estimates. Reliance on these erroneous estimates can have serious adverse consequences on the health of the person being monitored. For example, erroneous estimates can lead the person or his or her clinician to improperly interpret physiological state and cause the person to undergo treatment that is not medically indicated, or forego treatment that is medically indicated.

### SUMMARY OF THE INVENTION

The present invention provides a method and device for continual physiological monitoring in which the display of physiological parameter estimates is conditioned on conformance of the estimates with expectations. Current estimates of physiological parameters are compared with expectations for the current estimates determined using prior estimates of the physiological parameters. Nonconformance with expectations can result in display of information indicating present unavailability of an estimate for the physiological parameter. The method and device are adaptable for use with various types of monitored physiological parameters and various expectation metrics.

In one aspect of the invention, a method for continual physiological monitoring comprises acquiring by a physiological monitoring device a physiological signal; calculating by the device a current estimate of a physiological parameter from the physiological signal; evaluating by the device conformance of the current estimate with expectations for the current estimate determined by the device using one or more prior estimates of the physiological parameter calculated by the device from the physiological signal; and displaying by the device information regarding the current estimate determined by the device based at least in part on the evaluation.

In some embodiments, conformance of the current estimate with the expectations is determined based at least in part on whether the current estimate falls within a confidence interval for the current estimate.

In some embodiments, the confidence interval is a range whose midpoint is the most recent prior estimate.

In some embodiments, the confidence interval is a range whose midpoint is the second most recent prior estimate.

In some embodiments, the method further comprises recalculating by the device the most recent prior estimate as an average of the current estimate and the second most recent prior estimate.

In some embodiments, conformance of the current estimate with the expectations is determined based at least in part on whether the current estimate is higher than the most recent prior estimate and whether the most recent prior estimate is higher than the second most recent prior estimate.

In some embodiments, conformance of the current estimate with the expectations is determined based at least in part on whether the current estimate is lower than the most recent prior estimate and the most recent prior estimate is lower than the second most recent prior estimate.

In some embodiments, the displaying step comprises contemporaneously displaying by the device the current estimate and the most recent prior estimate.

In some embodiments, the displaying step comprises contemporaneously displaying by the device the most recent prior estimate and a trend arrow.

In some embodiments, the displaying step comprises displaying by the device an indication that the most recent prior estimate and the current estimate are presently unavailable.

In another aspect of the invention, a physiological monitoring device comprises a physiological data capture system; a physiological data acquisition system communicatively coupled with the capture system; a physiological data processing system communicatively coupled with the acquisition system; and a physiological data output interface communicatively coupled with the processing system, wherein the processing system receives a physiological signal from the capture system via the acquisition system, calculates a current estimate of a physiological parameter from the physiological signal, evaluates conformance of the current estimate with expectations for the current estimate determined using one or more prior estimates of the physiological parameter calculated from the physiological signal, and transmits to the output interface information regarding display of the current estimate determined based at least in part on the evaluation, whereupon information regarding the current estimate is displayed on the output interface.

These and other aspects of the invention will be better understood by reference to the following detailed description taken in conjunction with the drawings that are briefly described below. Of course, the invention is defined by the appended claims.

### BRIEF DESCRIPTION OF THE DRAWINGS

FIG. 1 shows a physiological monitoring device in some embodiments of the invention.

FIG. 2 shows consecutive sampling windows of a physiological signal in some embodiments of the invention.

FIG. 3 shows a normal distribution for a current estimate in some embodiments of the invention.

FIG. 4 shows a method for continual physiological monitoring in some embodiments of the invention.

FIGS. 5A-5C show display screens for displaying information regarding physiological parameter estimates in some embodiments of the invention.

### DETAILED DESCRIPTION OF A PREFERRED EMBODIMENT

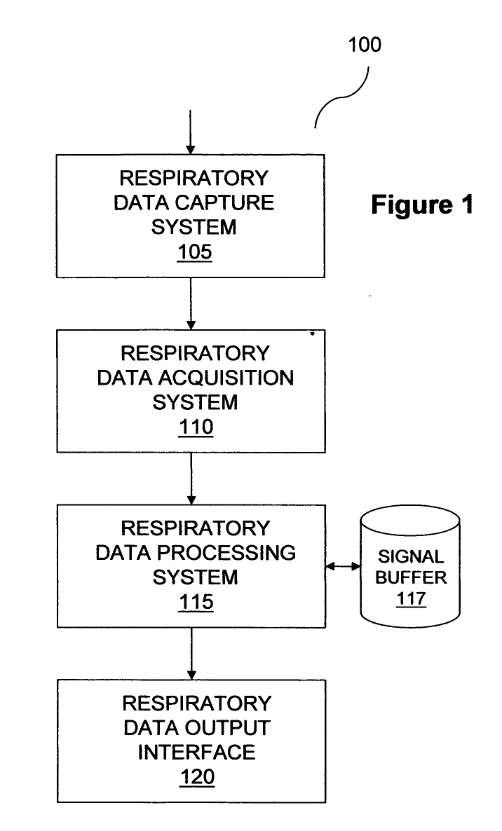

FIG. 1 shows a physiological monitoring device 100 in some embodiments of the invention. Monitoring device 100 includes a physiological data capture system 105, a physiological data acquisition system 110, a physiological data processing system 115 and a physiological data output interface 120 communicatively coupled in series. Processing system 115 is also communicatively coupled with a signal buffer 117.

Capture system 105 detects body sounds, such as heart and lung sounds, at a detection point, such as a trachea, chest or back of a person being monitored and transmits a physiological signal to acquisition system 110 in the form of an electrical signal generated from detected body sounds. Capture system 105 may include, for example, a sound transducer positioned on the body of a human subject.

Acquisition system 110 amplifies, filters, performs analog/digital (A/D) conversion and automatic gain control (AGC) on the physiological signal received from capture system 105, and transmits the physiological signal to processing system 115. Amplification, filtering, A/D conversion and AGC may be performed by serially arranged pre-amplifier, band-pass filter, final amplifier, ND conversion and AGC stages, for example.

Processing system 115, under control of a processor executing software instructions, processes the physiological signal to continually estimate one or more physiological parameters of the subject being monitored. Monitored physiological parameters may include, for example, heart rate and respiration rate. To enable continual estimation of physiological parameters, processing system 115 continually buffers in signal buffer 117 and evaluates samples of the physiological signal, wherein the length of each sample is equal to a sampling window length. Processing system 115 under control of the processor transmits to output interface 120 format and content information for displaying information regarding recent estimates of the monitored physiological parameters.

Output interface 120 includes a user interface having a display screen for displaying information in accordance with format and content information received from processing system 115 regarding recent estimates of physiological parameters. The displayed information may include, for example, the most recent prior estimate, the current estimate, trend arrows and indications that estimates are presently unavailable (e.g. question marks). Output interface 120 may also have a data management interface to an internal or external data management system that stores the information and/or a network interface that transmits the information to a remote monitoring device, such as a monitoring device at a clinician facility.

In some embodiments, capture system 105, acquisition system 110, processing system 115 and output interface 120 are part of a portable ambulatory monitoring device that monitors a person's physiological well-being in real-time as the person performs daily activities. In other embodiments, capture system 105, acquisition system 110, processing system 115 and output interface 120 may be part of separate devices that are remotely coupled via wired or wireless links.

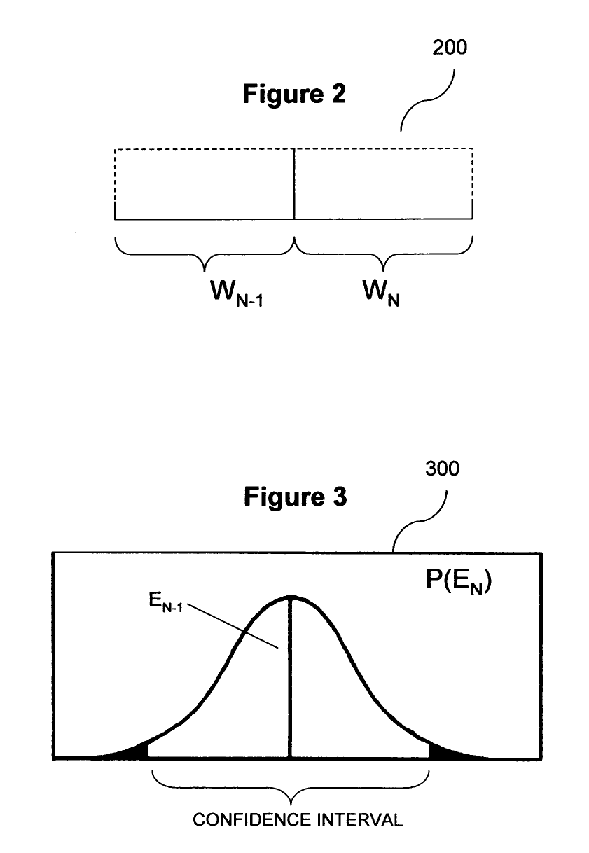

FIG. 2 shows consecutive sampling windows (W_{N-1}, W_{N}) 200 of a physiological signal in some embodiments of the invention. Each of the illustrated windows 200 is rectangular, such that data within the window is given equal weight. Moreover, the illustrated windows 200 are non-overlapping, although in other embodiments windows may be overlapping. Additionally, the illustrated windows 200 are of fixed length, although in other embodiments processing system 115 may dynamically adjust window length. Processing system 115 under control of a processor analyzes the signal data in windows (W_{N-1}, W_{N}) 200 to generate the most recent prior estimate E_{N-1} and the current estimate E_{N}, respectively, for one or more physiological parameters, such as heart rate or respiratory rate. The most recent prior estimate E_{N-1} and earlier prior estimates (e.g. E_{N-2}, E_{N-3}, E_{N-4}, etc.) are used by processing system 115 to determine expectations for the current estimate E_{N}, and the current estimate E_{N} is compared with its expectations to determine its acceptance and display status.

By way of example, one element of expectations for the current estimate E_{N} is conformance with a confidence interval for the current estimate calculated assuming a normal distribution. FIG. 3 shows a normal distribution P(E_{N}) for a current estimate E_{N} in some embodiments of the invention. The normal distribution P(E_{N}) is a bell-shaped curve having a midpoint at an expected mean for the current estimate E_{N} and a confidence interval having a range of plus or minus two standard deviations (+2σ) from the expected mean. If the current estimate E_{N} falls within the confidence interval, the current estimate E_{N} conforms to expectations and is accepted; otherwise, the decision of whether to accept the current estimate E_{N} is deferred pending additional analysis. For purposes of calculating the confidence interval for the current estimate E_{N}, the expected mean is set to the value of the most recent prior estimate E_{N-1}, and the standard deviation σ is set to a value calculated using the variance of a predetermined number of prior estimates (e.g. E_{N-1}, E_{N-2}, E_{N-3}, etc.) from their respective expected means (e.g. E_{N-2}, E_{N-3}, E_{N-4}, etc.).

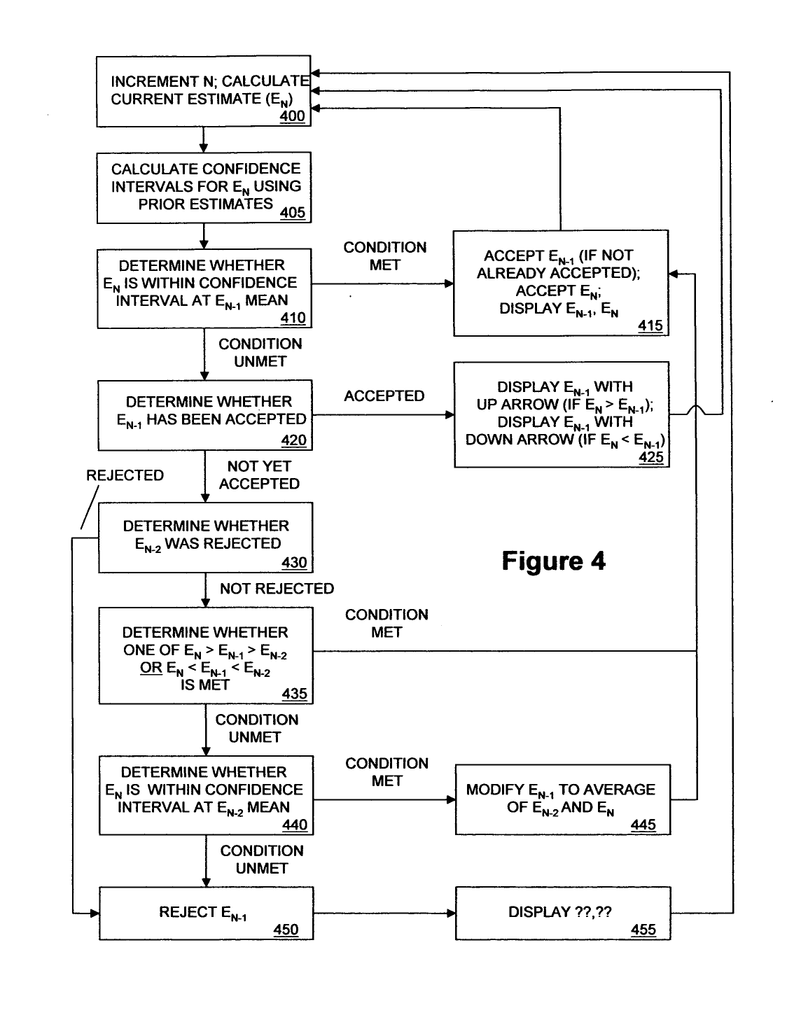

FIG. 4 shows a method for continual physiological monitoring in some embodiments of the invention. In these embodiments, the method is performed by processing system 115 under control of a processor that executes software instructions in conjunction with output interface 120 which displays information on a display screen in accordance with format and content information received from processing system 115 regarding recent estimates of a physiological parameter.

At Step 400, the next sample N is acquired and processing system 115 calculates the current estimate (E_{N}) from signal data in the sampling window (W_{N}).

At Step 405, processing system 115 calculates confidence intervals for the current estimate (E_{N}). The confidence intervals include a first confidence interval having a range of plus or minus two standard deviations (±2σ) from an expected mean at the most recent prior estimate (E_{N-1}), and a second confidence interval having a range of plus or minus two standard deviations (±2σ) from an expected mean at the second most recent prior estimate (E_{N-2}). In other embodiments, the ranges may span a smaller or larger number of standard deviations.

At Step 410, processing system 115 determines whether the current estimate (E_{N}) falls within the first confidence interval. That is, processing system 115 determines whether the current estimate (E_{N}) is within two standard deviations of the most recent prior estimate (E_{N-1}). If this condition is met, the current estimate (E_{N}) conforms to expectations and the flow proceeds to Step 415. If this condition is unmet, the flow proceeds to Step 420 for further analysis.

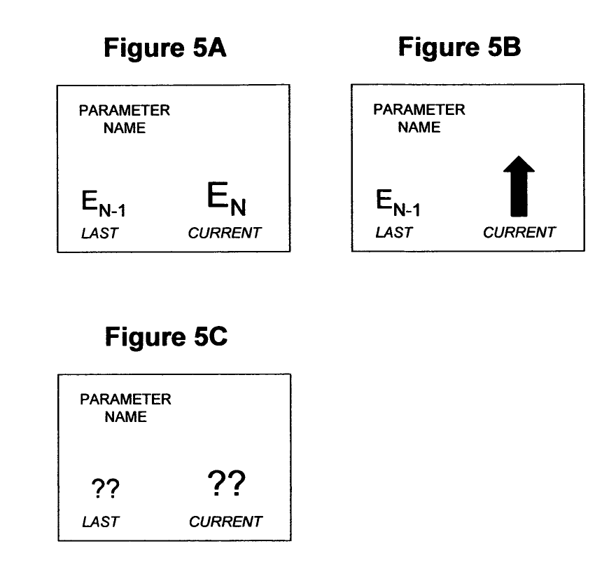

At Step 415, processing system 115 sets the acceptance status of the most recent prior estimate (E_{N-1}) to accepted (if not already set to accepted), sets the acceptance status of the current estimate (E_{N}) to accepted, and transmits information to output interface 120 instructing output interface 120 to contemporaneously display the most recent prior estimate (E_{N-1}) and the current estimate (E_{N}) in the format shown in FIG. 5A. The flow then returns to Step 400 where the next sample is considered.

At Step 420, processing system 115 determines whether the most recent prior estimate (E_{N-1}) has been accepted. If so, the decision on acceptance of the current estimate (E_{N}) is deferred and the flow proceeds to Step 425. If not, the flow proceeds to Step 430 for further analysis.

At Step 425, processing system 115 transmits information to output interface 120 instructing output interface 120 to contemporaneously display the most recent prior estimate (E_{N-1}) and a trend arrow in the format shown in FIG. 5B. The trend arrow is up if the current estimate (E_{N}) is greater than the most recent prior estimate (E_{N-1}) and the trend arrow is down if the current estimate (E_{N}) is less than the most recent prior estimate (E_{N-1}). The flow then returns to Step 400 where the next sample is considered.

At Step 430, processing system 115 determines whether the second most recent prior estimate (E_{N-2}) has been rejected. If so, the most recent prior estimate (E_{N-1}) will also be rejected and the flow proceeds to Step 450. If not, the flow proceeds to Step 435 for further analysis.

At Step 435, processing system 115 performs a sustained trend check to determine whether the current estimate (E_{N}) conforms with expectations even though it is outside the first confidence interval. In this check, processing system 115 determines whether either the current estimate (E_{N}) is part of a sustained upward trend in which the current estimate (E_{N}) is greater than the most recent prior estimate (E_{N-1}) which is in turn greater than the second most recent prior estimate (E_{N-2}) or, alternatively, the current estimate (E_{N}) is part of a sustained downward trend in which the current estimate (E_{N}) is less than the most recent prior estimate (E_{N-2}) which is in turn less than the second most recent prior estimate (E_{N-2}). If the current estimate (E_{N}) is part of a sustained upward or downward trend, the current estimate (E_{N}) conforms to expectations and the flow proceeds to Step 415. If the current estimate (E_{N}) is not part of a sustained upward or downward trend, the flow proceeds to Step 440 for further analysis.

At Step 440, processing system 115 performs a self-correction check to determine whether the current estimate (E_{N}) conforms to expectations even though it is outside the first confidence interval and is not part of a sustained upward or downward trend. In this check, processing system 115 evaluates whether the reason for nonconformance of the current estimate (E_{N}) with the first confidence interval is that the most recent prior estimate (E_{N-1}) was affected by a temporary adverse condition from which monitoring device 100 has since recovered, such as a temporary spike in signal noise or temporary sensor malfunction. Processing system 115 thus determines whether the current estimate (E_{N}) falls within the second confidence interval calculated in Step 405. That is, processing system 115 determines whether the current estimate (E_{N}) is within two standard deviations of the second most recent prior estimate (E_{N-2}). If this condition is met, the current estimate (E_{N}) conforms to expectations and the flow proceeds to Step 415 after recalculating the most recent prior estimate (E_{N-1}) at Step 445 as the average of the current estimate (E_{N}) and the second most recent prior estimate (E_{N-2}). If this condition is unmet, the flow proceeds to Step 450.

At Step 450, processing system 115 sets the acceptance status of the most recent prior estimate (E_{N-1}) to rejected, and transmits information to output interface 120 instructing output interface 120 to display an indication that the most recent prior estimate (E_{N-1}) and the current estimate (E_{N}) are presently unavailable as shown in FIG. 5C. The flow then returns to Step 400 where the next sample is considered.

It will be appreciated by those of ordinary skill in the art that the invention can be embodied in other specific forms without departing from the spirit or essential character hereof. The present description is therefore considered in all respects to be illustrative and not restrictive. The scope of the invention is indicated by the appended claims, and all changes that come with in the meaning and range of equivalents thereof are intended to be embraced therein.

What is claimed is:

1. A method for continual physiological monitoring, comprising:
acquiring by a physiological monitoring device a physiological signal;
calculating by the device a current estimate of a physiological parameter from the physiological signal;
evaluating by the device conformance of the current estimate with expectations for the current estimate determined by the device using one or more prior estimates of the physiological parameter calculated by the device from the physiological signal; and
displaying by the device information regarding the current estimate determined by the device based at least in part on the evaluation.

2. The method of claim 1, wherein conformance of the current estimate with the expectations is determined based at least in part on whether the current estimate falls within a confidence interval for the current estimate.

3. The method of claim 2, wherein the confidence interval is a range whose midpoint is the most recent prior estimate.

4. The method of claim 2, wherein the confidence interval is a range whose midpoint is the second most recent prior estimate.

5. The method of claim 4, further comprising recalculating by the device the most recent prior estimate as an average of the current estimate and the second most recent prior estimate.

6. The method of claim 1, wherein conformance of the current estimate with the expectations is determined based at least in part on whether the current estimate is higher than the most recent prior estimate and whether the most recent prior estimate is higher than the second most recent prior estimate.

7. The method of claim 1, wherein conformance of the current estimate with the expectations is determined based at least in part on whether the current estimate is lower than the most recent prior estimate and the most recent prior estimate is lower than the second most recent prior estimate.

8. The method of claim 1, wherein the displaying step comprises contemporaneously displaying by the device the current estimate and the most recent prior estimate.

9. The method of claim 1, wherein the displaying step comprises contemporaneously displaying by the device the most recent prior estimate and a trend arrow.

10. The method of claim 1, wherein the displaying step comprises displaying by the device an indication that the most recent prior estimate and the current estimate are presently unavailable.

11. A physiological monitoring device, comprising:
a physiological data capture system;
a physiological data acquisition system communicatively coupled with the capture system;
a physiological data processing system communicatively coupled with the acquisition system; and
a physiological data output interface communicatively coupled with the processing system, wherein the processing system receives a physiological signal from the capture system via the acquisition system, calculates a current estimate of a physiological parameter from the physiological signal, evaluates conformance of the current estimate with expectations for the current estimate determined using one or more prior estimates of the physiological parameter calculated from the physiological signal, and transmits to the output interface information regarding display of the current estimate determined based at least in part on the evaluation, whereupon information regarding the current estimate is displayed on the output interface.

12. The device of claim 11, wherein conformance of the current estimate with the expectations is determined based at least in part on whether the current estimate falls within a confidence interval for the current estimate.

13. The device of claim 12, wherein the confidence interval is a range whose midpoint is the most recent prior estimate.

14. The device of claim 12, wherein the confidence interval is a range whose midpoint is the second most recent prior estimate.

15. The device of claim 14, wherein the processing system recalculates the most recent prior estimate as an average of the current estimate and the second most recent prior estimate.

16. The device of claim 11, wherein conformance of the current estimate with the expectations is determined based at least in part on whether the current estimate is higher than the most recent prior estimate and whether the most recent prior estimate is higher than the second most recent prior estimate.

17. The device of claim 11, wherein conformance of the current estimate with the expectations is determined based at least in part on whether the current estimate is lower than the most recent prior estimate and the most recent prior estimate is lower than the second most recent prior estimate.

18. The device of claim 11, wherein the processing system contemporaneously displays the current estimate and the most recent prior estimate.

19. The device of claim 11, wherein the processing system contemporaneously displays the most recent prior estimate and a trend arrow.

20. The device of claim 11, wherein the processing system displays an indication that the most recent prior estimate and the current estimate are presently unavailable.
